(19) United States
(12) Patent Application Publication
(10) Pub. No.: US 2012/0035380 A1
(43) Pub. Date: Feb. 9, 2012

(54) PROCESS FOR PREPARING EPICHLOROHYDRIN FROM GLYCEROL

(76) Inventor: Walter Kanzler, Graz (AT)

(21) Appl. No.: 13/060,838

(22) PCT Filed: Aug. 24, 2009
(86) PCT No.: PCT/AT09/00326

§ 371 Date: Apr. 27, 2011

(30) Foreign Application Priority Data
Aug. 25, 2008 (AT) A 1309/2008

(51) Int. Cl. C07D 301/02

(52) U.S. Cl. 549/518

(57) ABSTRACT

A process for preparing epichlorohydrin includes chlorinating glycerol with gaseous hydrogen chloride in a bubble column, by supplying hydrogen chloride, glycerol and a mixture of monochloropropanediol and residues of glycerol recycled from the distillation units connected downstream of the bubble column, dichloropropanol and residues to the bottom of the bubble column, and, close to the top of the bubble column, drawing off the mixture of monochloropropanediol, dichloropropanol, water, residues of glycerol and residues formed there and feeding it to at least one distillation unit, after which the dichloropropanol and water distilled off there are hydrolyzed with sodium hydroxide solution. Unconverted hydrogen chloride drawn off from the top of the bubble column is absorbed with freshly supplied glycerol in a scrubber, and this hydrogen chloride-containing glycerol is supplied at the bottom of the bubble column. Preference is given to increasing the conversion rate by using oxalic acid as a catalyst.

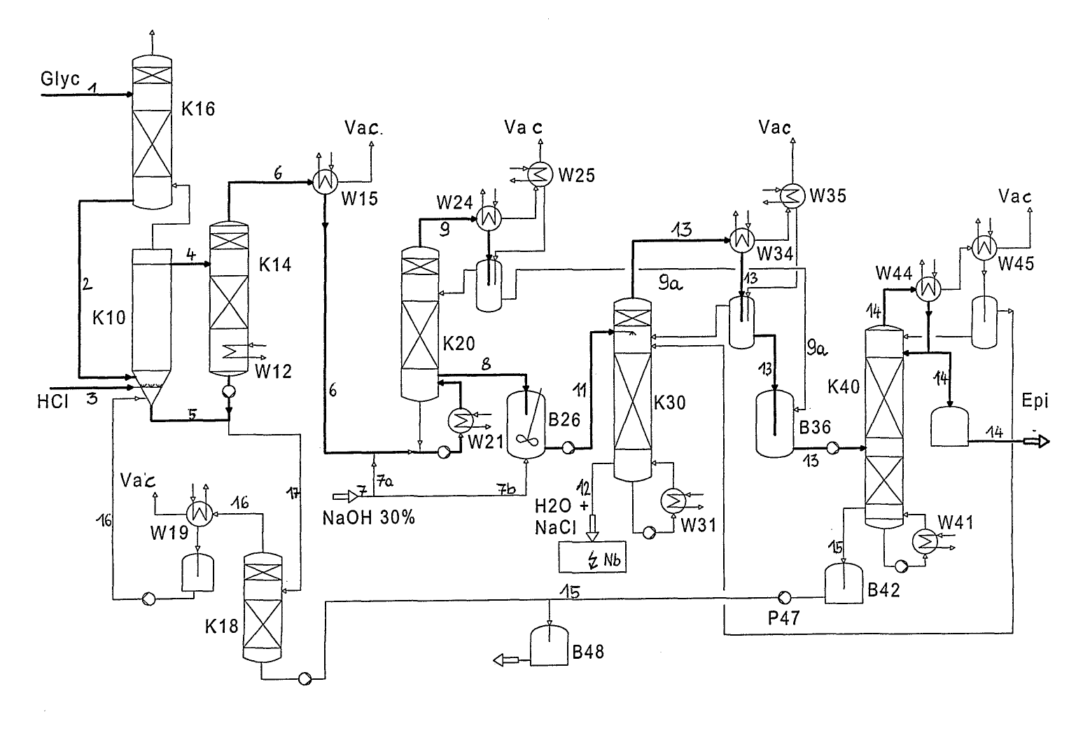

According to the precharacterizing clause of claim 1, the invention relates to a novel industrial process for the preparation of epichlorohydrin by chlorination of glycerol with hydrogen chloride in a reactor and distilling off of desired products in at least one distillation unit downstream of this, and subsequently hydrolysis of the dichloropropanol formed by means of sodium hydroxide solution.

In the cleavage and transesterification of triglycerides, glycerol, which is most suitable for chemical syntheses of the most diverse nature because of the high reactivity of its three OH groups, is formed in an amount of about 10% of the triglycerides employed.

DE 197308 B describes a process in the course of which glycerol is reacted with gaseous hydrogen chloride under increased pressure—under catalysis by means of acetic acid or propionic acid—to give dichloropropanol, an equilibrium with about 75% conversion being established.

DE 1 075 103 B describes a process in which, optionally in the presence of sulphurous acid in a one-stage distillation, an azeotrope of water and dichloropropanol is distilled off and hydrolysis is subsequently carried out with milk of lime and thereafter an azeotrope of epichlorohydrin and water is stripped off in a horizontal distillation tube. In this process, dichloropropanol can be obtained only with a yield of about 10% of the glycerol employed, and in the hydrolysis large amounts of calcium chloride are formed in a contaminated form as an environmentally unfriendly residue. WO 2005/021476 A1 describes the chlorination of glycerol and monochloropropanediol in a reactor cascade, catalysed by acetic acid, an azeotrope comprising water of reaction and some of the dichloropropanol produced being separated off. The majority of the dichloropropanol is fed back into the reactor again after a further distillation, and only the bottom product, comprising the residues, is sluiced out.

This process has the disadvantage that the top product of the distillation still contains relatively large amounts of hydrogen chloride and either the water distilled off can be condensed with the hydrogen chloride under a greatly reduced pressure at a low temperature only in a refrigerated unit, or the dichloropropanol formed in the reactor can be obtained as an azeotrope with a dichloropropanol content of only approx. 23% only with a considerable reflux of water into the top of the rectification column, which causes an enormous consumption of energy in the rectification.

The acetic acid employed as the catalyst is discharged out of the reaction mixture in the rectification because of its high vapour pressure, increases the consumption of alkali in the subsequent hydrolysis and contaminates the salt solution.

WO 2005/054167 A1 describes processes for the chlorination of various qualities of glycerol with contaminants from the preparation processes of glycerol. Acetic acid or another carboxylic acid, such as adipic acid or derivatives thereof, is proposed as the catalyst. The use of contaminated glycerols usually leads to deterioration in the yields and to the formation of large amounts of distillation residues, which in combination with chlorinated components can be disposed of only expensively and which are therefore preferably separated off before the chlorination.

The object of the present invention is thus to largely avoid the disadvantages of the processes known from the prior art, and to reduce to a minimum the energy input and the amount of residues to be disposed of.

The object according to the invention is thus to provide a process in which epichlorohydrin is prepared by chlorination of glycerol with hydrogen chloride and subsequent hydrolysis.

In order to avoid the abovementioned disadvantages, the novel process according to the abovementioned precharacterizing clause of claim 1 is characterized in that the glycerol is chlorinated with gaseous hydrogen chloride in co-current in a bubble column, wherein the hydrogen chloride, the glycerol and the mixture of chloropropanediol and residues of glycerol, dichloropropanol and residual substances which is fed back from a downstream distillation unit are fed into the bottom of the bubble column and a mixture essentially comprising monochloropropanediol, dichloropropanol, water, residues of glycerol and residual substances is taken off at the top of the bubble column and fed to a distillation unit, after which the dichloropropanol and water distilled off are hydrolysed, while the still unreacted residual hydrogen chloride taken off from the top of the bubble column is absorbed by the freshly fed glycerol in a washer.

According to claim 2, the hydrolysis of the dichloropropanol is advantageously carried out in two stages with aqueous sodium hydroxide solution, wherein in the first stage the sodium hydroxide solution is added in the stoichiometric ratio and the majority of the epichlorohydrin thereby formed is distilled off in a rectification column, and in a second stage, while topping up with sodium hydroxide solution, the remaining dichloropropanol is reacted practically completely to give epichlorohydrin and distilled off in a stripping column, preferably a rectification column, and the aqueous sodium chloride solution formed is employed, optionally after passing through a necessary after-purification stage, as the raw material for the chlor-alkali electrolysis for recovery of sodium hydroxide solution.

It has been found, surprisingly, that—as disclosed in claim 3—the use of oxalic acid as a catalyst in the bubble column produces the highest rates of reaction for the conversion of chloropropanediol into dichloropropanol, as a result of which the yield per unit time is comparatively considerably increased and at the same time the amount of residues and by-products formed is considerably lower than in the epichlorohydrin preparation processes disclosed hitherto.

In claim 5, the concentrations of oxalic acid in the bubble column which are most favourable for the processes are stated as being in the range of from 0.5 to 10 wt. %.

The use of a bubble column as a reactor for the chlorination is particularly advantageous, since the static pressure at the bottom of the bubble column, which is, for example, 5 to 15 m in height, increases the partial pressure of the hydrogen chloride, the higher solubility of this in the reactor liquid achieved in this manner considerably increasing the rate of reaction.

It has also been found that under a pressure at the top of the column of 1 bar absolute and at a liquid height of more than 5 m at reaction temperatures of between 100 and 130° C. hydrogen chloride practically almost no longer escapes at the top of the bubble column.

In the context of the invention, it has furthermore proved to be advantageous to wash out this small remaining amount of hydrogen chloride from the top of the bubble column in a wash column connected there using freshly fed, fully loadable glycerol—in this context see claim 5—in order to keep the hydrogen chloride content at the top and the content of organic (chlorine) compounds and the like as low as possible.

At the same time, for the rapid reaction it is important to take off liquid at the top of the bubble column and feed it into the downstream distillation unit in an amount and at a speed such that the equilibrium of the reaction is shifted as far as possible to the right towards the dichloropropanol. Since the chlorination to monochloropropanediol proceeds about 20 times as fast as the reaction to give dichloropropanol, it is furthermore important to keep the water concentration in the bubble column as low as possible and most preferably to separate off the reaction products formed from the reactor via a vacuum rectification column under 100 to 200 mbar absolute.

It is favourable or may be necessary to pass a small part of the bottom product of this rectification column over a further vacuum rectification in order to sluice out polymers and by-products in the bottom.

By this type of reaction procedure, dichloropropanol with a high purity which forms no by-products in the subsequent hydrolysis is obtained. As a result, the hydrolysis can be carried out to completion, and the azeotrope comprising epichlorohydrin and water can be separated off completely in the stripping columns—two downstream, provided in a manner which is favourable according to the invention—so that the remaining sodium chloride solution has a high purity and a concentration of more than >30 wt. %, depending on the concentration of the sodium hydroxide solution employed for the hydrolysis.

According to claim 6, the starting substances glycerol and hydrochloric acid, and glycerol, di- and monochloropropanol taken off from the rectification column are fed to the bubble column from the bottom, whereby a co-current of the reactants is achieved.

According to claim 7, a part stream of the bottom product from the rectification column downstream of the bubble column is passed, for separating off the dichloropropanol, into a further (vacuum) rectification column, in which chloropropanediol is separated off from the high-boiling components, such as, in particular, polymers, diglycerides and the like, and distilled off over the top, and where the high-boiling components are obtained as the distillation residue as the bottom product.

### EXAMPLE

Glycerol with a concentration of 99.5 wt. % with 0.4 wt. % of water and 0.1 wt. % of fatty acid residues is introduced with a material stream of 2,000 kg/h into the bubble column K10, at the bottom thereof, together with 1,560 kg/h of hydrogen chloride and the return runnings from the distillation and rectification columns K14 and K18. The bubble column K10 has e.g. a height of approx. 15 m and the filling level of the liquid in this is at about 10 m. The operating pressure in the bubble column is 1 bar abs. and the temperature there is kept at between 120 and 130° C. The hydrogen chloride introduced is reacted almost to the extent of 100%, the glycerol is converted into monochloropropanediol to the extent of 99% and the concentration of dichloropropanol in the bottom product is about 12 wt. %.

About 25,000 kg/h of liquid are taken off from the top of the bubble column K10, and in the downstream stripper K14 1,160 kg/h of water and about 2,346 kg/h of dichloropropanol are taken off over the top, and the bottom product thereof is fed back into the bubble column K10 to the extent of more than 90%, while the remaining 10% of the bottom product is fed to the vacuum rectification K18, at the top of which unreacted monochloropropanediol and the remaining dichloropropanol are taken off, while about 45 kg/h of by-products, mixed with about 25 kg of monochloropropanediol, are taken off from the bottom.

About 98% of the theoretical yield of dichloropropanol are taken off from the chlorination stage and fed to the hydrolysis in this manner.

The hydrolysis of the dichloropropanol is carried out with approx. 2,750 kg/h of 30 wt. % strength sodium hydroxide solution, and in the rectification column K20 approx. 1,900 kg/h of epichlorohydrin are taken off at the column head in the form of an aqueous azeotrope with a water content of about 26 wt. %, the azeotrope dissociating during the condensation into an aqueous phase and into an epichlorohydrin phase, the aqueous phase being introduced again as a reflux.

In the bottom of the column K20, the more than 30 wt. % strength sodium chloride solution is taken off with about 50 kg/h of dichloropropanol and residues of epichlorohydrin, and the hydrolysis is completed in the mixing stage B26 with the addition of a further 43 kg/h of 30 wt. % strength sodium hydroxide solution, and the salt solution formed there is fed to the further stripping column K30.

At the top of this column K30, an azeotrope of the remaining epichlorohydrin and water is likewise taken off, and in the bottom about 4,350 kg/h of about 28 wt. % strength sodium chloride solution with a content of less than 5 kg/h of chlorohydrocarbons are taken off, which are then additionally subjected to a treatment with ozone or hydrogen peroxide which is known per se and finally employed as the raw material in the chlor-alkali electrolysis, in order to obtain therefrom the sodium hydroxide solution (NaOH) envisaged for the hydrolysis.

The process according to the invention is explained in more detail with the aid of the drawing:

Via the washer K16, charged from the top of column K10 with small amounts of residual chlorine, glycerol—first freshly fed via line 1—loaded with small amounts of hydrogen chloride in the washer K16 is fed via line 2 to the bottom of the bubble reactor K10 and is introduced into this together with fresh hydrogen chloride fed in via line 3. Dichloropropanol, glycerol and the like obtained as the bottom product in the distillation column K14 downstream of the bubble column K10 is furthermore introduced there via line 5, and furthermore the dichloropropanol-containing top product of a parallel distillation column K18 supplied via line 17 with a part stream of the bottom product of the distillation column K14 is also introduced via line 16 for working up of residual amounts and residues obtained.

The top product of the bubble column K10 is introduced via line 4 into the first vacuum distillation column K14 already mentioned above, and from the top of this the mixture containing dichloropropanol as the desired product is introduced via line 6, after addition of 30 wt. % sodium hydroxide solution (NaOH) via lines 7, 7a, into the bottom of a further vacuum rectification column K20. Over the top of this, the epichlorohydrin-water mixture formed there is taken off via line 9.

From the bottom of the rectification column K20, sodium chloride solution and residual unreacted dichloropropanol is discharged, and after passing through line 8, it is treated and reacted in a mixing unit G26 with 30% strength aqueous NaOH fed in via line 7b—in a molar amount corresponding to the current content of dichloropropanol.

Thereafter, via line 11, the mixture formed there is fed into a first stripping column K30, from which, from the bottom thereof via line 12, aqueous sodium chloride solution is taken off, which is fed to a brine after treatment NB with chlor-alkali electrolysis.

From the top of the vacuum stripping column K30, crude epichlorohydrin is taken off via line 13 and collected in a container B36, into which a part stream of the epichlorohydrin-containing mixture already taken off at the top of the previous column K20 is also passed via line 9a. From there, the crude epichlorohydrin passes via line 13 here into a vacuum distillation column K40, over the top of which the desired pure epichlorohydrin is finally taken off via line 14.

Finally, from the bottom of the vacuum distillation column K40 just mentioned, the bottom products which have remained there are led into the abovementioned vacuum distillation column K18, from the top of which, via line 16, the bottom of the bubble column 10 is supplied with the mixture taken off there, the circulation thus being closed.

The essential advantages of the novel process operating with a bubble reactor and a distillation separated from this as the essential core apparatus lie in particular in the novel co-current feeding of glycerol and hydrogen chloride in the bubble reactor and in the equilibrium shifted towards the product side, in particular by the continuing taking off of the dichloropropanol formed and then of the epichlorohydrin to be distilled off.

There is also the surprising considerable increase in the rate of reaction at a level of at least 20% compared with that of the “best” processes known hitherto, which in particular is a consequence of the use of oxalic acid as a dibasic carboxylic acid instead of the organic acids employed hitherto as the catalyst, such as acetic acid, adipic acid and derivatives thereof.

1-7. (canceled)

8. A process for the preparation of epichlorohydrin by chlorination of glycerol with hydrogen chloride, the process comprising the following steps:
providing glycerol;
providing hydrogen chloride being catalysed by at least one carboxylic acid, in a reactor;
providing at least one distillation unit, the unit providings subsequent hydrolysis of dichloropropanol, the dichloropropanol formed by sodium hydroxide solution and glycerol chlorinated with gaseous hydrogen chloride in a bubble column;
feeding back the hydrogen chloride, glycerol and a mixture from the distillation unit, the distillation unit being downstream of a bubble column having monochloropropanediol, residues of glycerol, dichloropropanol, and residual substances;
feeding to the bottom of the bubble column and in the vicinity of the top of the bubble column, a mixture having monochloropropanediol, dichloropropanol, water, residues of glycerol, and residual substances;
taking off the mixture and feeding to at least one distillation unit,
distilling the dichloropropanol and water over the top of the distillation unit;
hydrolyzing the distillate from over the top of the distillation unit with sodium hydroxide solution,
optionally taking off hydrogen chloride from the top of the bubble column;
inhibiting reaction of the hydrogen chloride from the top of the bubble column;
absorbing the hydrogen chloride from the top of the bubble column with freshly fed glycerol in a washer; and
feeding the glycerol containing hydrogen chloride at the bottom of the bubble column.

9. The process according to claim 8, which further comprises carrying out the hydrolysis in two stages, including:
adding, in a first stage, sodium hydroxide solution (NaOH) in the stoichiometric ratio to the dichloropropanol coming from a first distillation unit;
forming by the adding of sodium hydroxide solution a portion of the total of epichlorohydrin formed;
distilling off the epichlorohydrin in a rectification column;
providing a second stage in which residual dichloropropanol is taken off from a bottom region of the rectification column;
converting by topping up of sodium hydroxide solution, substantially completely into epichlorohydrin;
distilling off the epichlorohydrin in a stripping column; and
combining, in a rectification column, distilled epichlorohydrin from the stripping column with the aqueous sodium chloride solution remaining there in a bottom region being employed, after passing through an after-treatment stage to allow raw material for chlor-alkali electrolysis for recovery of sodium hydroxide solution.

10. The process according to claim 8, wherein the oxalic acid is employed as a catalyst in the bubble column.

11. The process according to claim 8, wherein the concentration of oxalic acid in the bubble column is kept at 0.5 to 10 wt. %, based on the weight of the glycerol-hydrogen chloride mixture.

12. The process according to claim 8, wherein the concentration of oxalic acid in the bubble column is kept at 2 to 5 wt. %, based on the weight of the glycerol-hydrogen chloride mixture.

13. The process according to claim 8, wherein the residual amounts of hydrogen chloride at the top of the bubble column are absorbed in a washer downstream thereof by utilizing freshly fed glycerol, wherein glycerol pre-loaded with hydrogen chloride is fed to the bubble column, together with fresh hydrogen chloride, at the bottom of the bubble column.

14. The process according to claim 8, wherein initial glycerol and hydrogen chloride, and glycerol combined with di- and monochloropropanol are taken off from the rectification column are fed to the bubble column from the bottom.

15. The process according to claim 8, wherein a part of a stream of a bottom product from the rectification column is introduced, for separating off monochloropropanol contained there, into a second rectification column, where monochloropropanediol is separated of from the high-boiling components, being polymers, diglycerides and related compounds, which remain in the bottom of the second rectification column as a distillation residue, the monochloropropanediol is fed to the bottom of the bubble column.
